(19) United States
(12) Patent Application Publication
(10) Pub. No.: US 2021/0304861 A1
(43) Pub. Date: Sep. 30, 2021

(54) SYSTEM AND METHOD FOR ELIGIBLE PATIENT IDENTIFICATION, LEAKAGE QUANTIFICATION AND WORKFLOW SOFTWARE

(71) Applicants: Daniel Whitney STEVENSON, Quincy, MA (US); SHIELDS HEALTH MANAGEMENT COMPANY, LLC, Stoughton, MA (US)

(72) Inventor: Daniel Whitney STEVENSON, Quincy, MA (US)

(21) Appl. No.: 17/264,206

(22) PCT Filed: Aug. 15, 2019
(86) PCT No.: PCT/US2019/046695

§ 371 Date: Jan. 28, 2021

Related U.S. Application Data
(60) Provisional application No. 62/721,369, filed on Aug. 22, 2018.

(51) Int. Cl. G16H 20/10; G16H 10/60; G16H 40/20

(52) U.S. Cl. CPC G16H 20/10; G06Q 10/10; G16H 40/20; G16H 10/60

(57) ABSTRACT

The invention encompasses systems and methods to identify patients, and in particular eligible patients, of a health system or healthcare provider and gain insight, as to the areas of opportunity for a pharmacy to focus its efforts including, for example, a specific clinic within the hospital, a health insurance payer, or a limited distribution medication in order to improve care for the patients in greatest medical need as well as identify patients eligible to fill one or more medications at a particular pharmacy, whether a hospital-owned pharmacy or third-party pharmacy with whom the health system or healthcare provider has a contractual relationship.

### FIELD OF THE INVENTION

The invention encompasses systems and methods to identify patients of a health system or healthcare provider and gain insight as to the areas of opportunity for a pharmacy to focus its efforts in order to improve care for the patients in greatest medical need as well as identify patients eligible to fill one or more medications at a particular pharmacy.

### BACKGROUND OF THE INVENTION

The current state of the art involves a nurse, pharmacy technician or pharmacist monitoring a healthcare provider's electronic health records system for details on patients, including what medications the patients have been prescribed, and where they are currently filling their medications. Once the practitioner determine these details, they then begin a benefits investigation including a test claim to determine the patient's pharmacy benefit and whether the patient is eligible to fill a particular prescribed medication at a pharmacy.

The nurse, pharmacy technician or pharmacist uses the IT systems to run a benefits investigation, for example, a pharmacy's management system (e.g., Willow, Rx30, QS1), the health system's electronic medical records system (e.g., EPIC, Cerner, Allscripts), an eligibility portal (e.g., Emdeon or Nehan), a Medicaid eligibility finder (e.g., epaces, MassHealth portal) calling the patient's existing pharmacy, or ultimately calling the patient. Once the appropriate information is gathered, the pharmacy management system then conducts a test claim based on that patient's BIN, PCN, and Group number and the NDC code of the drug. This approach takes a substantial amount of time to complete (e.g., 15 minutes per patient and up to 60 minutes per patient), spread across many patients (in some instances >100 patients) per day for a single clinic makes this process untenable and does not allow for a user to fully understand the eligible patient roster of their clinic or disease state.

Further, this patient-by-patient approach invites errors, requires review of every patients' medications, medication regimen and insurance coming through the clinic. Ultimately many patients are missed and are not appropriately triaged to the pharmacy of the patient's choosing to receive the highest level of care and financially benefit the health system as well as the patient.

### SUMMARY OF THE INVENTION

The invention encompasses systems and methods to identify patients of a health system or healthcare provider and gain insight as to the areas of opportunity for a pharmacy to focus its efforts in order to improve care for the patients in greatest medical need as well as identify patients eligible to fill one or more medications at a particular pharmacy.

The invention generally encompasses a system and method wherein a user (e.g., a prescriber, nurse, case manager, pharmacy liaison, administrative staff, pharmacy technician or pharmacist) is presented with a list of one or more patients who are eligible to fill a prescription with the pharmacy of the user's or patient's choosing (including, but not limited to, a hospital-owned specialty pharmacy or third-party contract pharmacy, collectively “pharmacies”). In certain embodiments, the user is provided with the appropriate method (in-person vs. telephonically) to begin a discussion with the patient about the benefits of the pharmacies. This decision-tree logic regarding the most effective means to enroll the patient in a pharmacy program ensures the patient receives the highest level of care (as appropriate) and provides valuable insight to the user about the patient's medication adherence.

Accordingly, a first embodiment of the invention encompasses a system for identifying and matching patients to fill a prescription from a pharmacy comprising a server in communication with a user computing device or a plurality of user computing devices and one or more databases, wherein the server is configured to:
(a) receive a request from the user computing device or plurality of user computing devices to process one or more identifiable indicators of a patient comprising medical history, prescription medication history, personal health insurance information, or medication fulfillment history;
(b) in response to receipt of the request, retrieve data related to a patient, comprising prescription drug information, medical history, prescription medication history, personal health insurance information, medication fulfillment history and/or patient scheduling information from a database;
(c) match the retrieved data with a predetermined list of prescription drugs, physicians, clinics, and/or patient scheduling information;
(d) create an appointment list based on matches between drugs, physicians, clinics, and/or patient scheduling information in the predetermined lists;
(e) provide the matched data and a subset of the retrieved data to users to confirm eligibility of the patients; and
(f) determine whether or not the patient has a scheduled visit to a medical provider.

In an exemplary embodiment, the system for identifying and matching patients to fill a prescription includes the determining whether or not the patient has a scheduled visit to a medical provider and further includes:
(i) if the eligible patient has a scheduled visit, advising the user of a list of patients to meet at their visit to enroll the patient with a pharmacy; or
(ii) if the eligible patient does not have a scheduled visit within a pre-determined time period, advising the user to further communicate with a patient to enroll the patient with a pharmacy.

In another exemplary embodiment of the system for identifying and matching patients to fill a prescription, a list of patients eligible to fill a prescription using a hospital's pharmacy or pharmacy of the prescriber's choosing and who have been contacted in a given period is delivered to the user through one or more user interfaces.

In another exemplary embodiment of the system for identifying and matching patients to fill a prescription, one or more user interfaces provide contacted patients and uncontacted patient rosters and financial information to advise a health system of quality of patient care, medication adherence, and financial growth for the health system.

In another exemplary embodiment of the system for identifying and matching patients to fill a prescription, the pharmacy is a hospital-owned pharmacy or third-party pharmacy in which a health care system or healthcare provider has a contractual relationship.

In another exemplary embodiment of the system for identifying and matching patients to fill a prescription of claim 1, further comprising an algorithmic filtering process to process large amounts data from disparate systems including appointment data and electronic prescription data.

In another exemplary embodiment of the system for identifying and matching patients to fill a prescription, the data comprise healthcare providers' electronic medical record system, pharmacy management system data, and eligibility verification data.

In another exemplary embodiment of the system for identifying and matching patients to fill a prescription, includes a management system data that comprises data for patients who already fill prescriptions with the pharmacies.

In another exemplary embodiment of the system for identifying and matching patients to fill a prescription the eligibility verification data comprises confirmation that a patient's insurance covers the medication.

In another exemplary embodiment of the system for identifying and matching patients to fill a prescription, the user is a practitioner, clinician, prescriber, or administrator.

In another embodiment, the invention encompasses a method for identifying and matching patients to fill a prescription from a pharmacy comprising:
(a) receiving a request from the user computing device or plurality of user computing devices to process one or more identifiable indicators of a patient comprising medical history, prescription medication history, personal health insurance information, or medication fulfillment history;
(b) retrieving data related to a patient, comprising prescription drug information, medical history, prescription medication history, personal health insurance information, medication fulfillment history and/or patient scheduling information from a database;
(c) matching the retrieved data with a predetermined list of prescription drugs, physicians, clinics, and/or patient scheduling information;
(d) creating an appointment list based on matches between drugs, physicians, clinics, and/or patient scheduling information in the predetermined lists;
(e) providing the matched data and a subset of the retrieved data to users to confirm eligibility of the patients; and
(f) determining whether the patient has a scheduled visit to a medical provider.

In an exemplary embodiment, the method for identifying and matching patients to fill a prescription includes determining whether or not the patient has a scheduled visit to a medical provider and further comprises:
(i) advising the user of a list of patients to meet at their visit to enroll the patient with a pharmacy if the eligible patient has a scheduled visit, or
(ii) advising the user to further communicate with a patient to enroll the patient with a pharmacy if the eligible patient does not have a scheduled visit within a pre-determined time period.

In another exemplary embodiment of the method for identifying and matching patients to fill a prescription a list of patients eligible to fill a prescription using a hospital's pharmacy or pharmacy of the prescriber's choosing and who have been contacted in a given period is delivered to the user through one or more user interfaces.

In another exemplary embodiment of the method for identifying and matching patients to fill a prescription the one or more user interfaces provide contacted patients and uncontacted patient rosters and financial information to advise a health system of quality of patient care, medication adherence, and financial growth for the health system.

In another exemplary embodiment of the method for identifying and matching patients to fill a prescription the pharmacy is a hospital-owned pharmacy or third-party pharmacy in which a health care system or healthcare provider has a contractual relationship.

In another exemplary embodiment of the method for identifying and matching patients to fill a prescription further comprises an algorithmic filtering process to process large amounts data from disparate systems including appointment data and electronic prescription data.

In another exemplary embodiment of the method for identifying and matching patients to fill a prescription, the data comprise healthcare providers' electronic medical record system data, pharmacy management system data, and prior authorization data.

In another exemplary embodiment of the method for identifying and matching patients to fill a prescription, the pharmacy management system data comprises data for patients who already fill prescriptions with the pharmacies.

In another exemplary embodiment of the method for identifying and matching patients to fill a prescription, wherein the prior authorization data comprises confirmation that a patient's insurance covers the medication.

In another exemplary embodiment of the method for identifying and matching patients to fill a prescription, wherein the user is a practitioner, clinician, prescriber, or administrator.

### BRIEF DESCRIPTION OF THE FIGURES

FIG. 1 illustrates a flow chart illustrating an exemplary processing method of the invention

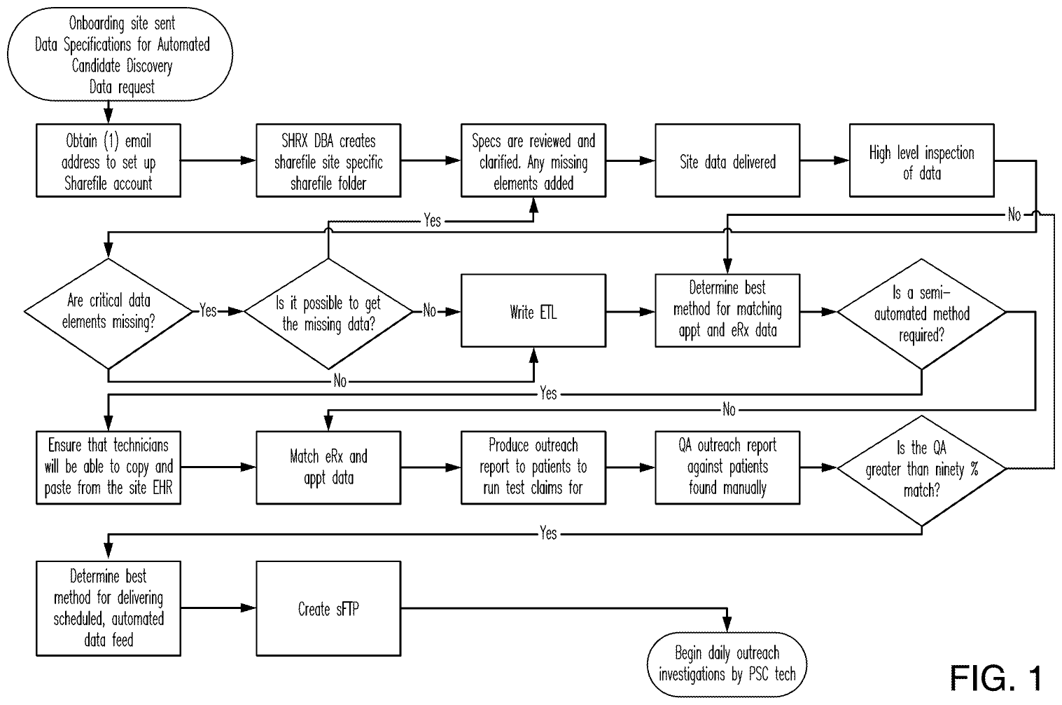

FIG. 2 illustrates a flow chart illustrating an exemplary processing method of the invention.

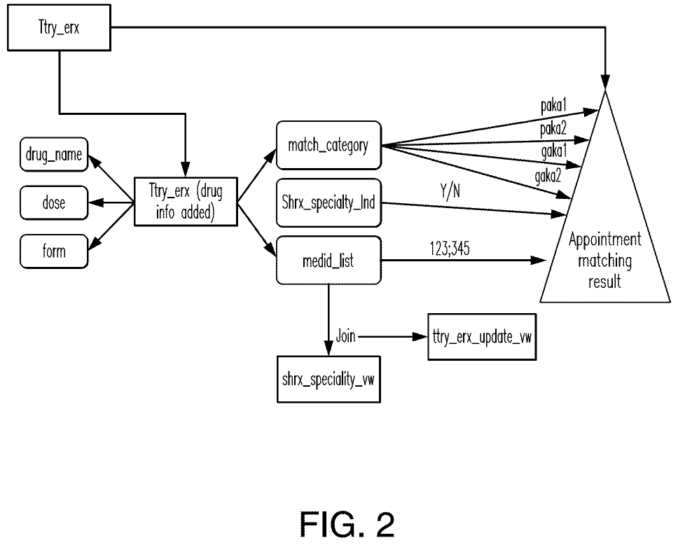

FIG. 3 illustrates a flow chart illustrating an exemplary processing method of the invention.

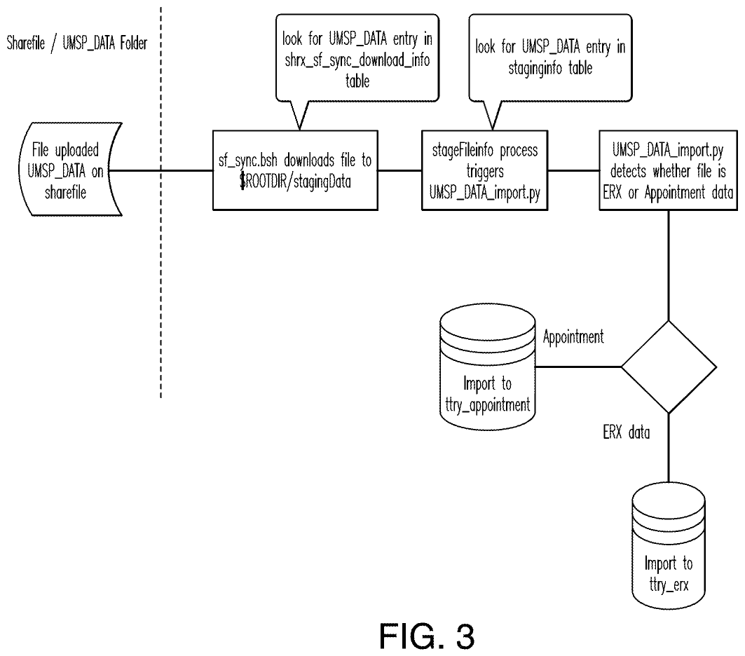

FIG. 4 illustrates a flow chart illustrating an exemplary processing method of the invention.

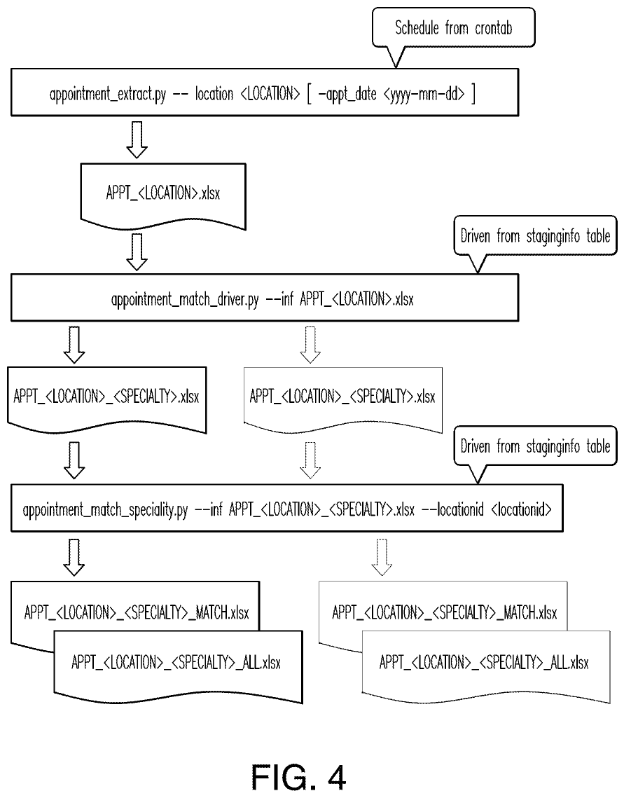

### DETAILED DESCRIPTION OF THE INVENTION

The invention generally encompasses systems and methods for identifying and matching patients to fill a prescription from a pharmacy comprising a server in communication with a user computing device or a plurality of user computing devices and one or more databases, wherein the server is configured to:
(a) receive a request from the user computing device or plurality of user computing devices to process one or more identifiable indicators of a patient comprising medical history, prescription medication history, personal health insurance information, or medication fulfillment history;
(b) in response to receipt of the request, retrieve data related to a patient, comprising prescription drug information, medical history, prescription medication history, personal health insurance information, medication fulfillment history and/or patient scheduling information from a database;
(c) match the retrieved data with a predetermined list of prescription drugs, physicians, clinics, and/or patient scheduling information;
(d) create an appointment list based on matches between drugs, physicians, clinics, and/or patient scheduling information in the predetermined lists;
(e) provide the matched data and a subset of the retrieved data to users to confirm eligibility of the patients; and
(f) determine whether or not the patient has a scheduled visit to a medical provider.

In exemplary embodiments, the system and method for identifying and matching patients to fill a prescription includes the determining whether or not the patient has a scheduled visit to a medical provider and further includes:
(i) if the eligible patient has a scheduled visit, advising the user to meet that patient at his or her visit to enroll the patient with a pharmacy; or
(ii) if the eligible patient does not have a scheduled visit within a pre-determined time period, advising the user to communicate with a patient to enroll the patient with a pharmacy.

In another exemplary embodiment, the system and methods for identifying and matching patients to fill a prescription, a list of patients eligible to fill a prescription using a hospital's pharmacy or pharmacy of the prescriber's choosing and who have been contacted in a given period is delivered to the user through one or more user interfaces.

In one exemplary embodiment, a prescriber, nurse, pharmacy technician or pharmacist (exemplary embodiments of “users”) opens a software solution and is presented with a single list of patients who may be eligible to fill a prescription with a pharmacy of the user's choosing including, but not limited to, the hospital-owned specialty pharmacy and third-party contract pharmacies, (collectively “Pharmacies”).

In another exemplary embodiment, the user is provided with the appropriate communication method (for example in-person vs. telephonically) to begin a discussion with the patient about the benefits of the Pharmacies.

In certain embodiments, this decision-tree logic provides the most effective means to enroll the patient in the Pharmacy program, ensures the patient receives the highest level of care (as appropriate), and provides valuable insight to the user about the patient's medication adherence.

In certain embodiments, eligible patients may be identified using various embodiments of the invention. In one exemplary embodiment, a user (e.g., pharmacy technician, clinic liaison, or registered nurse) begins by opening a PSC module and a list of eligible patients with either an upcoming appointment or an action to engage telephonically the patient who receives care from a particular doctor(s)/clinic (based on previous user input and/or the system configuration) and a list of medications determined by the system (based on cost and complexity of the medication) for whom the user should investigates benefits, determining patient's BIN, PCN, and Group Number based on inputting the information on a patient including, but not limited to, a patient's first and last name, date of birth, gender, and/or last four digits of their social security number.

In another embodiment, a user clicks on a patient name and runs a test claim (in an automated or manual manner) to determine whether the pharmacy of the prescriber's choice can fill the medication for the patient or not (e.g., if the patient was precluded from filling with that pharmacy as a result of payer lockout or from limited distribution drugs (LDDs)). In another embodiment, the user confirms the copay amount as well as the varying day's supply offered by the identified pharmacy and can fill for via the pharmacy management system based on a contractual agreement with a pharmacy benefit manager.

In certain embodiments, a successful test claim results in patient data being populated on the Liaison/Health System user module and added to an eligible patient roster for the health system. In certain embodiments, the user, upon opening the module, is presented with a list of patients to meet at an appointment or call telephonically based on the decision tree logic incorporated in the solution. In certain embodiments, the user will then meet the patient when they come into the user's clinic to visit a clinician/prescriber. In certain embodiments, the user, if not the clinician, confirms with the clinician in advance of the appointment or telephonic outreach that the user may contact the patient. In certain embodiments, once user meets with or calls the patient, the user enters the details of the meeting, whether the patient agreed to enroll with the pharmacy of the prescriber's choosing or not and the rationale.

In other embodiments, the method of creating a list of patients eligible to fill with a hospital's pharmacy or pharmacy of the prescriber's choosing and who have been contacted in a given period can be delivered through a variety of user interfaces including a software user interface; a spreadsheet; a .doc, .pdf, .csv, .txt, or other similar static electronic outputs; or paper printouts.

In certain embodiments, the invention can also provide the eligible/uncontacted eligible patient rosters and financial opportunity provided in a user interface or flat file to help the health system understand a holistic view of where they can make the greatest impact on patient care, medication adherence, and financial growth for the health system.

In certain embodiments, the algorithmic filtering process ingests large amounts data from disparate systems including appointment data and electronic prescription data from the healthcare providers' electronic medical record system, pharmacy management system data (for patients who already fill with the Pharmacies), and prior authorization data (to ensure the patient's insurance covers the medication). In certain embodiments, the decision-tree algorithm determines whether the user should meet the new eligible patient at an in-person meeting or telephonically.

As used herein, the term “prior authorization” refers to any of a series of checks and confirmations including data created by manual and/or automated benefits investigation and test claims, which are conducted by insurance companies and/or third party payers before they will agree to cover certain prescribed medications or medical procedures.

The instant prior authorization invention is intended among other things to overcome and avoid the costly and time-consuming nature of the current state of prior authorization.

In certain embodiments, the system's matching process, which uses electronic prescription data for patients previously prescribed a particular medication (part of a specific medication list that evolves based on a set or parameters including medical complexity, price, risk of adverse event, and difficulty to administer or ship), such medication is identified based on its National Drug Code, Data Vendor ID, or text match from a proprietary medication formulary.

In other embodiments, the addition to the specific drug, the matching process filters based on the doctor who prescribed the medication (provided that doctor is a member of a set of pre-determined doctors or clinics, as identified by each doctor's National Provider Identifier or clinic's location name). In certain embodiments, this is all done programmatically using algorithms to ensure accuracy and precise patient targeting. In certain embodiments, during the implementation process, lists of targeted drugs, physicians, clinics, and patients are developed in collaboration with the health system along with unique alpha-numerical identifiers for each entity on each of the lists (e.g., a National Provider Identifier (“NPI”) for physicians), such alpha-numerical identifier is verified present in the ingested source data such that it can then be matched on an ongoing basis to populate the prioritized appointment and telephonic outreach list for users. In certain embodiments, an illustrative algorithmic filtering process organizes the matching of the above criteria such that processing performance is optimized, beginning with the greatest reductions in the data entries of the source data and ending with the lowest. If no matches are found, the algorithm triggers a quality assurance “check” process whereby it signals interventions from a user to ensure data integrity and availability.

In other embodiments, the primary advantages of the invention are simplicity, efficiency, and robustness. In certain embodiments, using a data-driven approach to eligible patient identification delivers a robust process that is able to identify substantially all of the eligible patients that can be enrolled by a hospital's pharmacy or pharmacy of the prescriber's choosing, without missing a large portion of patients coming into a hospital. The invention has advantages over the state of the art as the art does not use comprehensive data sets or algorithms to determine a refined set of patients for whom the user should investigate pharmacy benefits and run test claims and is a currently a very manual process. In certain embodiments, the typical user is overwhelmed with information from many sources (often siloed) while our invention simplifies the information overload by combining multiple data sources into one source of truth and filtering out superfluous information as well as medications and transferring noisy data into specific patients to enroll. Based on over two years of utilization, roughly 3% of patient appointments for a particular clinic are patients that are eligible to fill with one of the Pharmacies. In certain embodiments, the ability to know the 3% and have the user be prompted to engage those patients is otherwise unfeasible given the tedious work required, user bandwidth and required data aggregation from various sources.

In other embodiments, the invention is used/leveraged to provide robust leakage and lost opportunity reports to help health system/healthcare providers know which clinics, disease states, medications and payers provide the greatest opportunity to engage and enroll patients in the pharmacy of their choosing. In certain embodiments, this ensures that the patient receives the highest level of care and therefore achieves above-industry-standard levels of medication adherence and financial benefit to the health system in a simple and regulatory-compliant manner. In certain embodiments, this also provides the health system/healthcare provider with the insights to know which payers and drug manufactures the Pharmacies are currently locked out of/ineligible to fill for. In other embodiments, the system and methods provide a road map to ensure that the patients the health system/healthcare providers care for receive the gold standard in medication therapy management through identifying which payers and drug manufactures to engage in network access conversations to be able to enroll those patients in the pharmacy of their choosing as well as the ability to manage the healthcare provider's staffing/headcount, thereby ensuring efficiency and labor cost control.

As used herein, the term “system” includes, for example, a bus or other communication mechanism for communicating information, and a processor coupled with bus for processing information. By way of example, the computer system may be implemented with one or more processors. Processor may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. A system can include, in addition to hardware, code that creates an execution environment for the computer program in question for example, code that constitutes processor firmware, a protocol stack, a database management system, an operating system, or a combination of one or more of them stored in an included memory, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to a bus for storing information and instructions to be executed by processor. The processor and the memory can be supplemented by, or incorporated in, special purpose logic circuitry.

The instructions may be stored in the memory and implemented in one or more computer program products, i.e., one or more modules of computer program instructions encoded on a computer readable medium for execution by, or to control the operation of, the system, and according to any method well known to those of skill in the art, including, but not limited to, computer languages such as data-oriented languages (e.g., SQL, dBase, PostgreSQL), system languages (e.g., C, Objective-C, C++, Assembly), architectural languages (e.g., Java, Java Springboot, .NET), and application languages (e.g., PHP, Ruby, Perl, Python, Elm). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi-paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, Wirth languages, embeddable languages, and xml-based languages. Memory may also be used for storing temporary variable or other intermediate information during execution of instructions to be executed by processor.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

A system further includes a data storage device such as a magnetic disk or optical disk, coupled to bus for storing information and instructions. A system may be coupled via input/output module to various devices. The input/output module can be any input/output module. Example input/output modules include data ports such as USB ports. The input/output module is configured to connect to a communications module. Example communications modules include networking interface cards, such as Ethernet cards and modems. In certain aspects, the input/output module is configured to connect to a plurality of devices, such as an input device and/or an output device. Example input devices include a keyboard and a pointing device, e.g., a mouse or a trackball, by which a user can provide input to the computer system. Other kinds of input devices can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices include display devices, such as a LED (light emitting diode), CRT (cathode ray tube), or LCD (liquid crystal display) screen, for displaying information to the user.

According to one aspect of the present disclosure, the execution of the sequences of instructions contained in main memory causes processor to perform the process steps described herein. One or more processors in a multi-processing arrangement may also be employed to execute the sequences of instructions contained in memory. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a personal area network (PAN), a local area network (LAN), a campus area network (CAN), a metropolitan area network (MAN), a wide area network (WAN), a broadband network (BBN), the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

A system can include clients and servers. A client and server are generally remote from each other and typically interact through a communication network. The relationship of client and server arises by virtue of computer programs running on the respective computers and having a client-server relationship to each other. A system can be, for example, and without limitation, a desktop computer, laptop computer, or tablet computer. A system can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer readable medium” as used herein refers to any medium or media that participates in providing instructions or data to processor for execution. Such a medium may take many forms, including, but not limited to, non-volatile media, volatile media, and transmission media. Non-volatile media include, for example, optical disks, magnetic disks, or flash memory, such as data storage device 406. Volatile media include dynamic memory, such as memory. Transmission media include coaxial cables, copper wire, and fiber optics, including the wires that comprise bus. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” The term “some” refers to one or more. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

### EXAMPLES

### Illustrative Example 1

In one exemplary embodiment, a user (e.g., a clinician) interacts with liaison regarding a patient introduction or urgent task (including PA request) is the #1 priority:

New patient teach appointments, if scheduled;

Respond to patient calls immediately;

Otherwise, if Liaison has appointment within next 15 minutes, then Liaison should go into TRx and EMR to review patient info and find patient in pre-agreed (e.g., with clinic) meeting area, have patient interaction, and log results in TRx

If there is no appointment/physician interaction:

Liaison reviews emails, EMR messages, & pending and recently completed PAs and FAs from PA log each day at a specified time;

Liaison interacts with clinicians then checks e-mails and EMR messages;

Liaison reviews e-mails, EMR messages, & pending and recently completed PAs and FAs from PA log at the end of each day;

Liaison discusses next day's appointments and any new telephonic outreach patients on the clean list with clinic contact at the end of each day;

Depending on number of Refill/Therigy Calls with due date <Today+10 days:
1. Reach out to patients about whom an external pharmacy calls (or faxes) liaison to discuss (proactive outreach), immediately after receiving the call (or fax) from the external pharmacy, and then log the result in TRx;
2. Otherwise, if there are Refill/Therigy Calls with due date <Today+10 days, then make 1 refill call to each of those patients, logging results in Therigy and PMS as appropriate, then complete delivery slip and arrange logistics in PMS for connected patients;

Once there are 0 refill calls due (i.e., #2 supra completed) then reach out to any patients with PAs expiring within 4 weeks (for potential proactive outreach) and log the results in TRx;

Once there are zero patients with expiring PAs, complete any urgent patient requests, PAs, or FAs that the PSC is not going to work on;

Contact each patient on telephonic outreach list, logging results in TRx, and process in PMS as needed;

Respond to any new patient, clinician, or company team e-mails;

Check in with patients on service with hospital specialty pharmacy whose medication were delivered yesterday (last business day) to coordinate/confirm medication delivery, where needed;

Contact clinicians asking if any patients have been having trouble with their copays, receiving their medications, or adhering to their medications;

Make refill calls for patients who have due dates within 11-14 days and log results in Therigy and EMR, as appropriate.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The subject matter of this specification has been described in terms of particular aspects, but other aspects can be implemented and are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other variations are within the scope of the following claims.

What is claimed is:

1. A system for identifying and matching patients to fill a prescription from a pharmacy comprising:
a server in communication with a user computing device or a plurality of user computing devices and one or more databases, wherein the server is configured to:
(a) receive a request from the user computing device or plurality of user computing devices to process one or more identifiable indicators of a patient comprising medical history, prescription medication history, personal health insurance information, or medication fulfillment history;
(b) in response to receipt of the request, retrieve data related to a patient, comprising prescription drug information, medical history, prescription medication history, personal health insurance information, medication fulfillment history and/or patient scheduling information from a database;
(c) match the retrieved data with a predetermined list of prescription drugs, physicians, clinics, and/or patient scheduling information;
(d) create an appointment list based on matches between drugs, physicians, clinics, and/or patient scheduling information in the predetermined lists;
(e) provide the matched data and a subset of the retrieved data to users to confirm eligibility of the patients; and
(f) determine whether or not the patient has a scheduled visit to a medical provider.

2. The system for identifying and matching patients to fill a prescription of claim 1, wherein the determining whether or not the patient has a scheduled visit to a medical provider further comprises:
(i) if the eligible patient has a scheduled visit, advising the user of a list of patients to meet at their visit to enroll the patient with a pharmacy; or
(ii) if the eligible patient does not have a scheduled visit within a pre-determined time period, advising the user to further communicate with a patient to enroll the patient with a pharmacy.

3. The system for identifying and matching patients to fill a prescription of claim 2, wherein a list of patients eligible to fill a prescription using a hospital's pharmacy or pharmacy of the prescriber's choosing and who have been contacted in a given period is delivered to the user through one or more user interfaces.

4. The system for identifying and matching patients to fill a prescription of claim 3, wherein the one or more user interfaces provide contacted patients and uncontacted patient rosters and financial information to advise a health system of quality of patient care, medication adherence, and financial growth for the health system.

5. The system for identifying and matching patients to fill a prescription of claim 1, wherein the pharmacy is a hospital-owned pharmacy or third-party pharmacy in which a health care system or healthcare provider has a contractual relationship.

6. The system for identifying and matching patients to fill a prescription of claim 1, further comprising an algorithmic filtering process to process large amounts data from disparate systems including appointment data and electronic prescription data.

7. The system for identifying and matching patients to fill a prescription of claim 6, wherein the data comprise healthcare providers' electronic medical record system, pharmacy management system data, and eligibility verification data.

8. The system for identifying and matching patients to fill a prescription of claim 7, wherein the management system data comprises data for patients who already fill prescriptions with the pharmacies.

9. The system for identifying and matching patients to fill a prescription of claim 7, wherein the eligibility verification data comprises confirmation that a patient's insurance covers the medication.

10. The system for identifying and matching patients to fill a prescription of claim 1, wherein the user is practitioner, clinician, prescriber, or administrator.

11. A method for identifying and matching patients to fill a prescription from a pharmacy comprising:
(a) receiving a request from the user computing device or plurality of user computing devices to process one or more identifiable indicators of a patient comprising medical history, prescription medication history, personal health insurance information, or medication fulfillment history;
(b) retrieving data related to a patient, comprising prescription drug information, medical history, prescription medication history, personal health insurance information, medication fulfillment history and/or patient scheduling information from a database;
(c) matching the retrieved data with a predetermined list of prescription drugs, physicians, clinics, and/or patient scheduling information;
(d) creating an appointment list based on matches between drugs, physicians, clinics, and/or patient scheduling information in the predetermined lists;
(e) providing the matched data and a subset of the retrieved data to users to confirm eligibility of the patients; and
(f) determining whether the patient has a scheduled visit to a medical provider.

12. The method for identifying and matching patients to fill a prescription of claim 11, wherein the determining whether or not the patient has a scheduled visit to a medical provider further comprises:
(i) advising the user of a list of patients to meet at their visit to enroll the patient with a pharmacy if the eligible patient has a scheduled visit, or
(ii) advising the user to further communicate with a patient to enroll the patient with a pharmacy if the eligible patient does not have a scheduled visit within a pre-determined time period.

13. The method for identifying and matching patients to fill a prescription of claim 12, wherein a list of patients eligible to fill a prescription using a hospital's pharmacy or pharmacy of the prescriber's choosing and who have been contacted in a given period is delivered to the user through one or more user interfaces.

14. The method for identifying and matching patients to fill a prescription of claim 13, wherein the one or more user interfaces provide contacted patients and uncontacted patient rosters and financial information to advise a health system of quality of patient care, medication adherence, and financial growth for the health system.

15. The method for identifying and matching patients to fill a prescription of claim 11, wherein the pharmacy is a hospital-owned pharmacy or third-party pharmacy in which a health care system or healthcare provider has a contractual relationship.

16. The method for identifying and matching patients to fill a prescription of claim 11, further comprising an algorithmic filtering process to process large amounts data from disparate systems including appointment data and electronic prescription data.

17. The method for identifying and matching patients to fill a prescription of claim 16, wherein the data comprise healthcare providers' electronic medical record system, pharmacy management system data, and prior authorization data.

18. The method for identifying and matching patients to fill a prescription of claim 17, wherein the management system data comprises data for patients who already fill prescriptions with the pharmacies.

19. The method for identifying and matching patients to fill a prescription of claim 17, wherein the prior authorization data comprises confirmation that a patient's insurance covers the medication.

20. The method for identifying and matching patients to fill a prescription of claim 11, wherein the user is practitioner, clinician, prescriber, or administrator.
